(19) United States
(12) Patent Application Publication
(10) Pub. No.: US 2025/0125035 A1
(43) Pub. Date: Apr. 17, 2025

(54) AUTO-CALCULATING NUTRIENT TRACKING SYSTEM AND METHOD

(71) Applicants: Jacob Bockelmann, Santa Rosa, CA (US); Andres Matias Bordese, Córdoba (AR)

(72) Inventors: Jacob Bockelmann, Santa Rosa, CA (US); Andres Matias Bordese, Córdoba (AR)

(21) Appl. No.: 18/486,977

(22) Filed: Oct. 13, 2023

(51) Int. Cl. G16H 20/60

(52) U.S. Cl. CPC G16H 20/60

(57) ABSTRACT

The present invention introduces an advanced nutrient tracking system that operates on mobile and other computing devices. Users interact with the system through a mobile application, which allows for the input of personal goals and food data entries. The system features a computational engine capable of dynamically adjusting serving sizes and nutrient values based on user-defined targets. The computational engine also recalculates scaling factors when a user modifies a nutrient value, thereby providing real-time, customized nutrient tracking. The system enables users to customize the desired nutrients for tracking.

### CROSS-REFERENCE TO RELATED APPLICATIONS

N/A

### BACKGROUND OF THE INVENTION

### 1. Field of the Invention

The present invention relates to dietary software but more particularly to an auto-calculating nutrient tracking system and method.

### 2. Description of Related Art

The importance of proper nutrition in maintaining a healthy lifestyle is well understood. However, the task of tracking and managing nutrient intake can be cumbersome and time-consuming. Traditional methods of nutrient tracking often involve manual calculations, referencing various food databases, and using generic meal planning tools that lack customization options. These methods are not only inefficient but also prone to errors, making it difficult for individuals to adhere to specific dietary goals or medical recommendations.

Several software applications and platforms have been developed to assist users in tracking their nutrient intake. However, these existing solutions primarily focus on post-consumption tracking and do not offer comprehensive features for meal preparation. Furthermore, they often lack the capability to target specific nutrients, thereby limiting their utility for users with specialized dietary needs or health conditions.

A significant limitation of current systems is the lack of user customization for each nutrient. Most existing platforms offer a one-size-fits-all approach, which does not account for individual dietary needs or preferences. Additionally, these systems often fail to provide proper scaling of serving sizes, making it challenging for users to accurately measure their nutrient intake. The inability to customize other nutrients, such as vitamins and minerals, further restricts the effectiveness of these platforms.

### BRIEF SUMMARY OF THE INVENTION

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

In one aspect, a nutrient tracking system for dynamically adjusting serving sizes and nutrient values is provided, the system comprising an application software executable on one or more computing devices, wherein the application software executes a graphical user interface on the one or more computing devices; a data repository for storing food data entries and user-defined nutrient targets, wherein the food data entries includes meals comprised of one or more food elements, each food element having a serving size and nutrient values for a plurality of nutrients; a computational engine configured to: receive user inputs for specific nutrient value targets within a food element of the one or more food elements; calculate a scaling factor based on the user inputs; adjust the serving size and nutrient values of the food element in real-time based on the calculated scaling factor; recalculate the scaling factor when a user modifies a nutrient value within a food element; and, update the graphical user interface with the updated nutrient values and serving size.

In another aspect, a nutrient tracking system for modifying base food element data, the system comprising an application software executable on one or more computing devices, wherein the application software executes a graphical user interface on the one or more computing devices; a data repository for storing base food element data for a plurality of food elements, daily food data entries comprised of at least one food element of the plurality of food elements, and user-defined nutrient targets, wherein each base food element of the base food element data includes a serving size and nutrient values for a plurality of nutrients; the graphical user interface configured to: allow a user to select a base food element from the plurality of food elements; enable the user to modify any individual nutrient value within the selected base food element; wherein the modification is isolated to the individual nutrient value and does not affect other nutrient values within the selected base food element or its serving size.

In one embodiment, the system further comprises a computational engine configured to: receive user nutrient value inputs for specific nutrient targets within a food element of the plurality of food elements of a daily food data entry; calculate a scaling factor based on the user inputs; adjust the serving size and nutrient values of the food element in real-time based on the calculated scaling factor; recalculate the scaling factor when a user modifies a nutrient value within a food element; wherein the user interface is also configured to update the graphical user interface with the updated nutrient values and serving size; and wherein said modification of any individual nutrient value of the daily food data entry does not affect other nutrient values within the selected base food element or its serving size.

In yet another aspect of the invention, a method for nutrient tracking and dynamic adjustment of serving sizes and nutrient values is provided, the method comprising the steps of executing an application software on one or more computing devices, wherein the application software provides a graphical user interface; storing food data entries and user-defined nutrient targets in a data repository, wherein the food data entries include meals comprised of one or more food elements, each food element having a serving size and nutrient values for a plurality of nutrients; receiving, via the graphical user interface, user inputs for specific nutrient value targets within a food element of the one or more food elements; calculating a scaling factor based on the user inputs; adjusting the serving size and nutrient values of the food element in real-time based on the calculated scaling factor; recalculating the scaling factor when a user modifies a nutrient value within a food element; updating the graphical user interface with the updated nutrient values and serving size; allowing a user to select a base food element from a plurality of food elements stored in the data repository; enabling the user to modify any individual nutrient value within the selected base food element; wherein the modification is isolated to the individual nutrient value and does not affect other nutrient values within the selected base food element or its serving size.

The foregoing has outlined rather broadly the more pertinent and important features of the present disclosure so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present disclosure. It should be realized by those skilled in the art that such equivalent structures do not depart from the spirit and scope of the invention as set forth in the appended claims.

### BRIEF DESCRIPTION OF THE SEVERAL VIEWS OF THE DRAWINGS

Other features and advantages of the present invention will become apparent when the following detailed description is read in conjunction with the accompanying drawings, in which:

FIG. 1 is a network diagram of the auto-calculating nutrient tracking system according to an embodiment of the present invention.

FIG. 2 is a system architectural diagram of the auto-calculating nutrient tracking system according to an embodiment of the present invention.

FIG. 3 is a flow diagram of the auto-calculating nutrient tracking method according to an embodiment of the present invention.

FIG. 4 is a flow diagram of the auto-calculating nutrient tracking method according to an embodiment of the present invention.

FIG. 5 is a user interface of the auto-calculating nutrient tracking system according to an embodiment of the present invention.

FIG. 6a is a user interface of the auto-calculating nutrient tracking system and method according to an embodiment of the present invention.

FIG. 6b is a user interface of the auto-calculating nutrient tracking system and method according to an embodiment of the present invention.

FIG. 7a is a user interface of the auto-calculating nutrient tracking system and method according to an embodiment of the present invention.

FIG. 7b is a user interface of the auto-calculating nutrient tracking system and method according to an embodiment of the present invention.

FIG. 8 is a system architectural diagram of the auto-calculating nutrient tracking system according to an embodiment of the present invention.

### DETAILED DESCRIPTION OF THE PREFERRED EMBODIMENT

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein to specifically provide an auto-calculating nutrient tracking system and method.

It is to be understood that the terminology used herein is for the purpose of describing particular embodiments only and is not intended to be limiting. The terms “a” or “an,” as used herein, are defined as to mean “at least one.” The term “plurality,” as used herein, is defined as two or more. The term “another,” as used herein, is defined as at least a second or more. The terms “including” and/or “having,” as used herein, are defined as comprising (i.e., open language). The term “providing” is defined herein in its broadest sense, e.g., bringing/coming into physical existence, making available, and/or supplying to someone or something, in whole or in multiple parts at once or over a period of time. These terms generally refer to a range of numbers that one of skill in the art would consider equivalent to the recited values (i.e., having the same function or result). In many instances these terms may include numbers that are rounded to the nearest significant figure. The term “real-time” is defined as updates occurring instantaneously within a time frame of less than 5 seconds depending on processing speed of the user device.

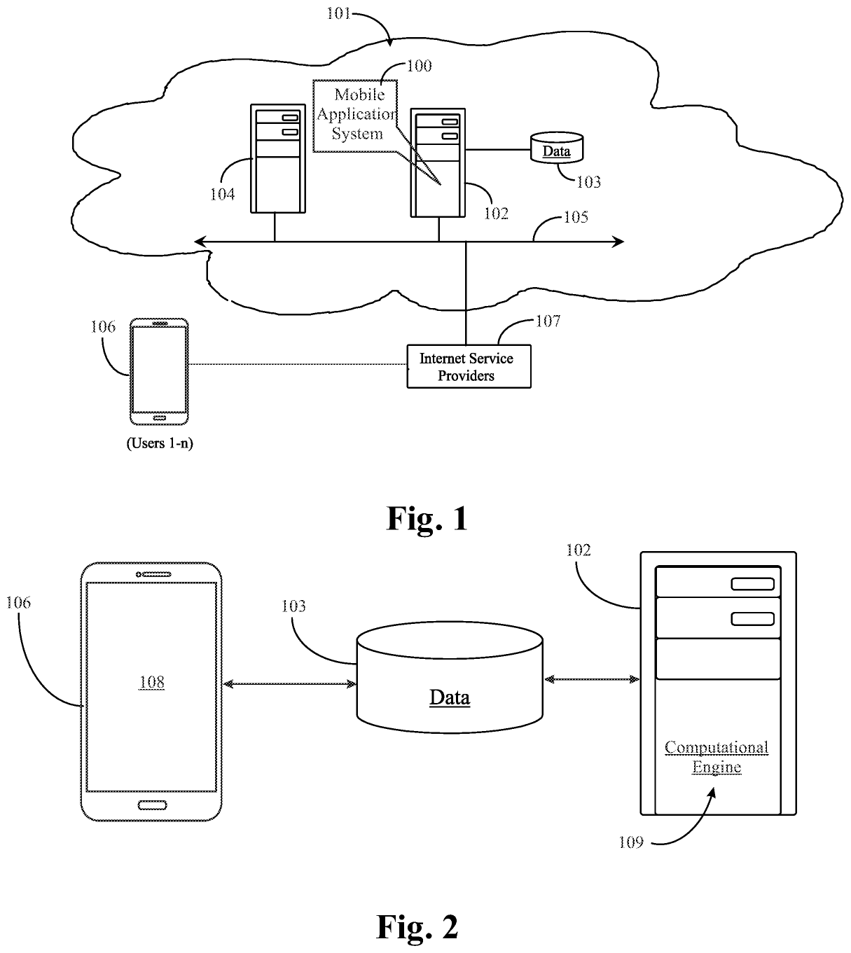

FIG. 1 is a network diagram of the auto-calculating nutrient tracking system according to an embodiment of the present invention. Referring to FIG. 1, the system comprises one or more internet-connected central servers 102 executing application software 100 from non-transitory media. A central server 102 is connected to a data repository 103, which may be any sort of data storage known in the art. The system further comprises a third party internet-connected server 104 connected to internet backbone 105. Although one third party internet-connected server 104 is shown, it is understood that potentially millions of other similar servers are connected to the internet via internet backbone 105. A number of users, via computing devices 106, are connected to the internet-connected central server 102 via an internet service provider (ISP) 107, allowing the number of users to access the application software 100, and download the software on their computing device 106. Advantageously, the present invention enables the number of users to utilize most functionalities of the auto-calculating nutrient tracking system without a data or Internet connection. In some embodiments, the computing device 106 is a mobile device. In other embodiments, the computing device is a laptop or desktop computer.

FIG. 2 is a system architectural diagram of the auto-calculating nutrient tracking system according to an embodiment of the present invention. Referring now to FIG. 2, a mobile device 106 having a graphical user interface (GUI) 108 is illustrated in communication with data repository 103 and central server 102. In some embodiments, the mobile device may be any internet connected computer device, including but not limited to a smartphone, tablet, or desktop computer. The data repository 103 may include local storage (on the mobile device), storage hosted via the central server 102, and/or storage provided by a third party server, such as cloud storage, dedicated or shared. The communication between these components is capable of two-way communication. The GUI 108 serves as the primary means for user interaction, enabling users to input information such as age, weight, and dietary goals (e.g., weight loss, weight gain, etc.). The GUI 108 also allows users to add or import food data entries, create meals, and adjust serving sizes and nutrient values dynamically. In one embodiment, the system includes a computational engine 109 that is configured and capable of dynamically adjusting serving sizes and other nutrient values based on user inputs for specific nutrient targets. The computational engine 109 can be hosted by the central server 102 and/or be available on the user device's local storage, enabling offline use.

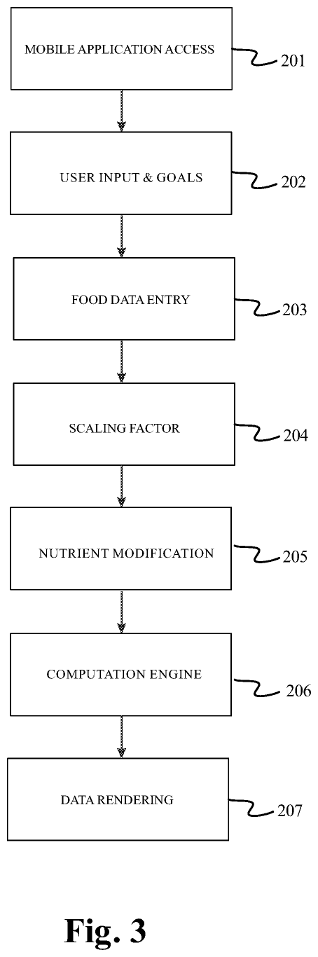

FIG. 3 is a flow diagram of the auto-calculating nutrient tracking method according to an embodiment of the present invention. Referring now to FIG. 3, the method comprises steps, starting with step 201, a user accesses the system via the mobile application (100; FIG. 1). It should be understood; the term mobile application is not limiting and although the system is generally accessed via a mobile device, any computing device may be used. In step 202, the user is prompted to input information and goals, such as measurables, age, weight, activity level, and dietary goals: e.g. weight loss, weight gain, etc. In step 203, the user may add or import food data entries into the system. These food data entries can be manually added by the user or imported via the data repository 103 hosted by the server 102. More specifically, the user may create base food elements, meals, and recipes representing a user's daily intake of food consumption, wherein meals and recipes are a collection of one or more base food elements. These base food elements may be manually added to the system by the user or imported via the data repository 103 hosted by the server 102. In some embodiments, the available base food elements for importing may be based on crowd sourced data from other users, or added to the system by other means, as well known in the art. For example, in some embodiments, the system supports automatic data import from third-party databases or devices, such as barcode scanners.

Once the base food elements are added, a user has the ability to adjust the scaling factor when adding foods to a meal, via step 204, if desired. More specifically, each food data entry has an associated implicit scaling factor, which defaults to 1. The scaling factor indicates the multiplier for the base serving size to determine the actual serving consumed. For example, if the default serving size of peanut butter is 2 tbsp, a scaling factor of 10 implies the consumption of 20 tbsp. After the scaling factor is determined based on the default service size of the base food element data, a nutrient modification 205 occurs. In some embodiments, a nutrient modification occurs when a user adjusts a nutrient value within a food element. When this occurs, in step 206, the computation engine computes a new scaling factor based on the specific adjustment to the nutrient value. Finally, in step 207, the data is rendered and the new values are outputted and displayed to the user.

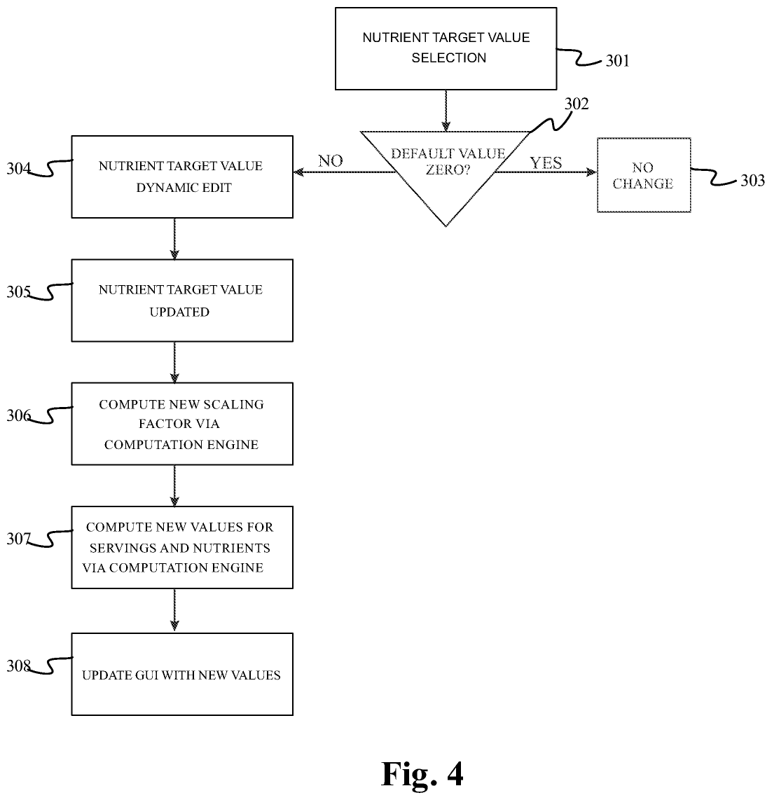

Referring now to FIG. 4, a more detailed flow diagram of the auto-calculating nutrient tracking method is illustrated. In 301, a nutrient target value selection is engaged by a user, wherein the nutrient value is toggled for editing. In 302, if the default value is zero, no change occurs in step 303, i.e. the system ignores the input or provides a warning or error message. If the default value is not zero, in 304, the nutrient value is able to be dynamically edited by the user. In 305, the nutrient value is updated. In 306, a new scaling factor is computed via the computation engine, wherein the scaling factor is determined by the scale or value entered by the user compared to the default value. In 307, the computation engine recomputes new values for the serving size and other nutrients based on the scaling factor. In 308, the graphical user interface (GUI) is updated with the new values.

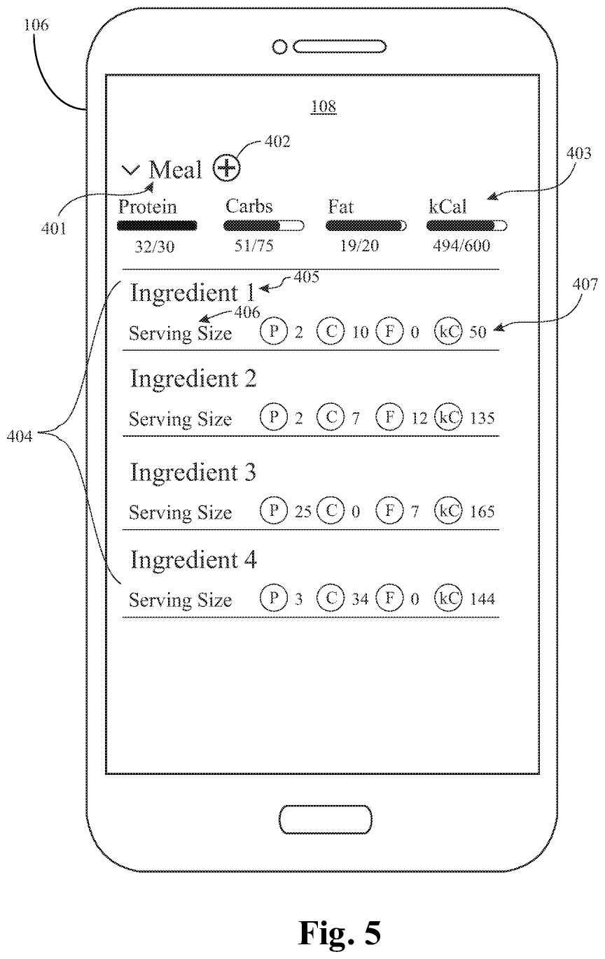

FIG. 5 is a graphical user interface of the auto-calculating nutrient tracking system. Referring now to FIG. 5, the graphical user interface (GUI) 108 of the user device 106 is illustrated. In this exemplary instance, the GUI 108 displays a meal 401 having at least one food 404, wherein the at least one food is a base food element the meal is comprised of. The meal may be any collection of food elements, such as breakfast, lunch, dinner, snack, etc. The user has the ability to add or remove foods to the meal, such as with additional element 402, which prompts the user to add a base food element from local storage, or importing food data elements as previously discussed. The user's nutrient targets 403 are illustrated such that the visual progress (goals) are shown based on each individual target nutrient, or macronutrients, including but not limited to protein, carbohydrates, fat, as well as calories, cholesterol, fiber, water, sodium, saturated fat, sugar, unsaturated fat, vitamins, iron, potassium, calcium, minerals and other nutrients. It should be noted that this is not an exhaustive list, and other nutrients may be provided by the system depending on the specific target goals desired by the user. Further, the user is enabled to add and customize the types of nutrients they wish to track and/or be displayed. In some embodiments, the visual progress goals are shown by bar graph, pie graph, or other visual representation to show progress as well known in the art. In some embodiments, specific values, fractions, percentages, etc. based on the progress is also listed, enabling the user to see what is required to meet their goals.

Still referring to FIG. 5, each food 405, includes the serving size value 406, and the specific nutrient values 407 for that service size of that specific food 405. In the example shown, protein, carbohydrates, fats, and kilocalories (calories) are illustrated, but these are not limited, and other nutrients may be displayed. Although, not illustrated, in some embodiments, total daily nutrient targets may be displayed, which is met with a plurality of meals, each comprised of a number of foods or base food elements.

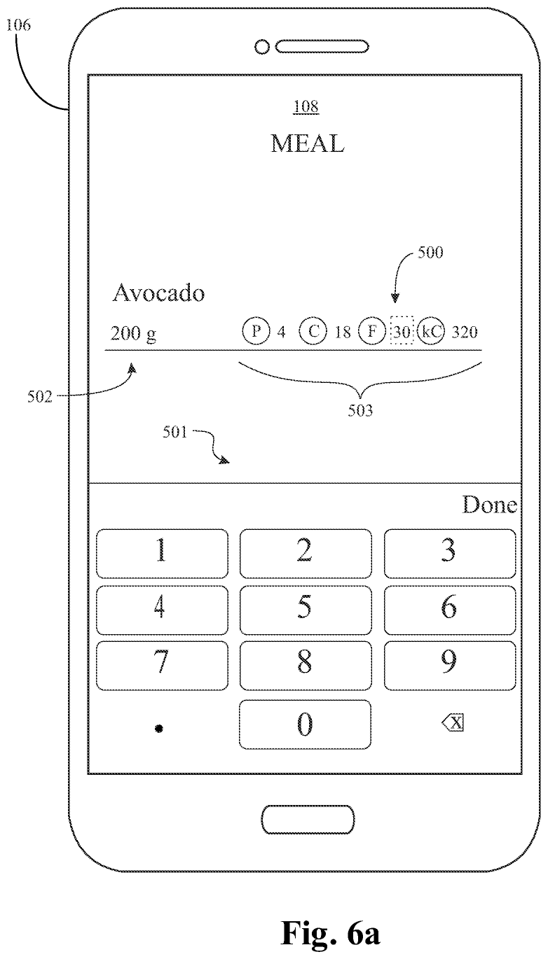

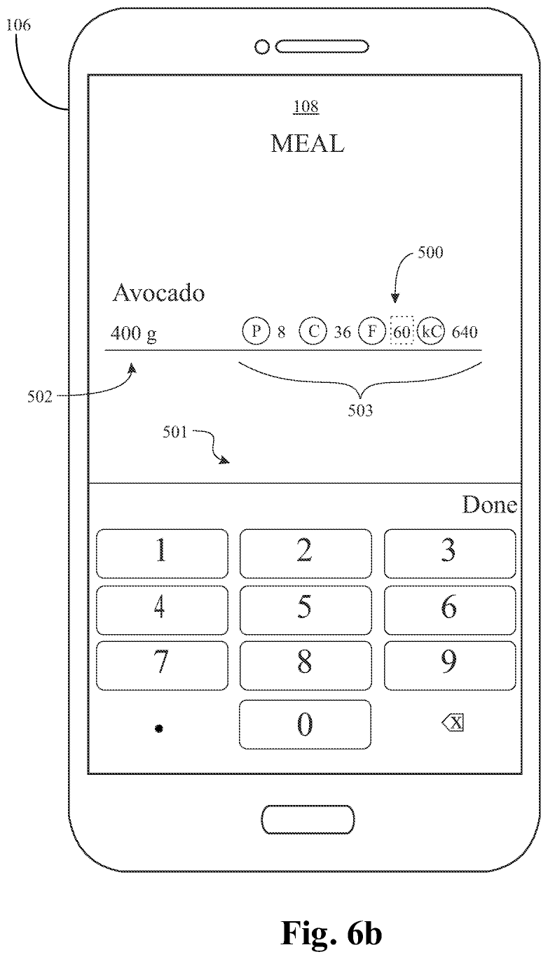

FIGS. 6a-b illustrate a graphical user interface 108 exemplary instance of a nutrient modification 500 and scaling factor via the computation engine on a user device 106. Referring now to FIGS. 6a-b, in one embodiment, a user has a meal including one food, in this case Avocado, having a serving size of 200 g. It should be understood that the serving size may be in any unit of measure as desired by the user, which varies based on the type of food. In some embodiments, this may be more of a quantifiable amount, teaspoon, tablespoons, cups, grams, ounces, etc. or may be units or quantities, such as slices, pieces, etc. For the specific service size, a plurality of nutrients values 503 are displayed. These are determined by the base food data stored in the data repository as previously discussed.

Advantageously, a user has the ability to modify any nutrient value in the plurality of nutrient values 503 via a nutrient modification 500. In the exemplary instance shown in FIG. 6a, the fat nutrient value is selected, wherein in this example, the default fat nutrient value is “30” for a 200 g serving size. In one embodiment, the user is configured to use a tap gesture as well known in the art of smartphone and touch screens to toggle the selection. In other types of computing devices, any known selection techniques may be used. Once the desired nutrient value is selected, the user may enter in a new desired value for that nutrient using a numerical keyboard 501. In this example, referring now to FIG. 6b, the user doubles the fat nutrient value to “60”, once this value is entered, and “done” is selected, the computing engine performs the recomputation process to compute a new scaling factor. In one embodiment the new scaling factor is calculated via the following formula: New Scaling Factor=User Input Nutrient Value/Default Nutrient Value Per Serving. Thus, the new scaling factor is “2” and each nutrient value and serving size is recalculated. There is no limit to the amount of nutrient adjustments possible. This is advantageous to the user as it dynamically adjusts the remaining nutrients/serving size to answer the question, “How much X do I need to eat to get Y?”, wherein X is the serving size and Y is any specific or collection of nutrient values. Other nutrient tracking systems of the prior art ask the user ‘How much of X did you eat?’ and then return the resulting Y nutrients. The computing engine of the present invention provides a significant advantage over other nutrient tracking system of the prior art, as it prevents the user from trying to determine the correct portions or serving sizes of the foods or meals to fit specific macronutrient or nutrient goals, which is a difficult and error prone process, thus improving the user's ability to meet and track their goals accurately. This struggle to find the correct foods in the correct serving sizes to fit one's nutrition targets is known in the art as playing “macro tetris.” It should be noted that adjustments to any nutrient values in this stage, does not affect the base food element data. This will be discussed in more detail below.

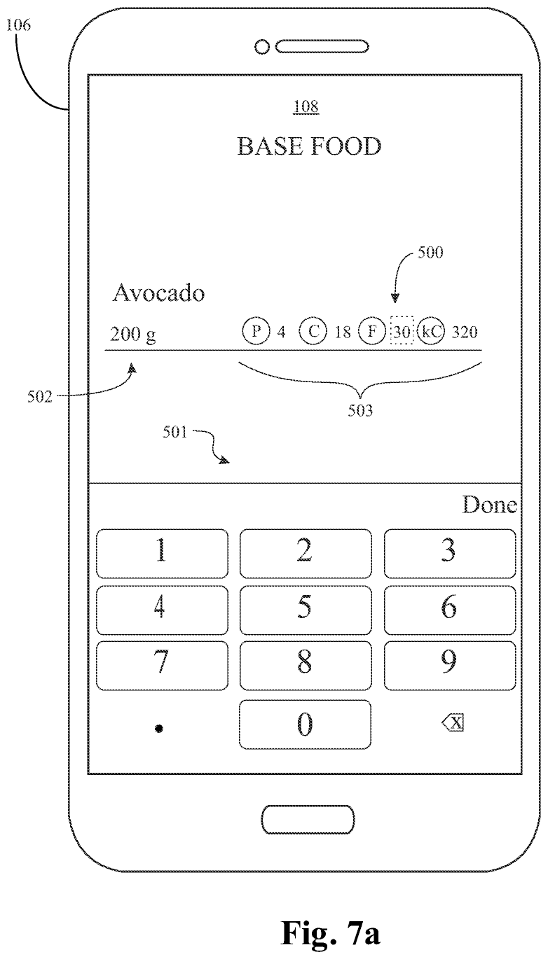

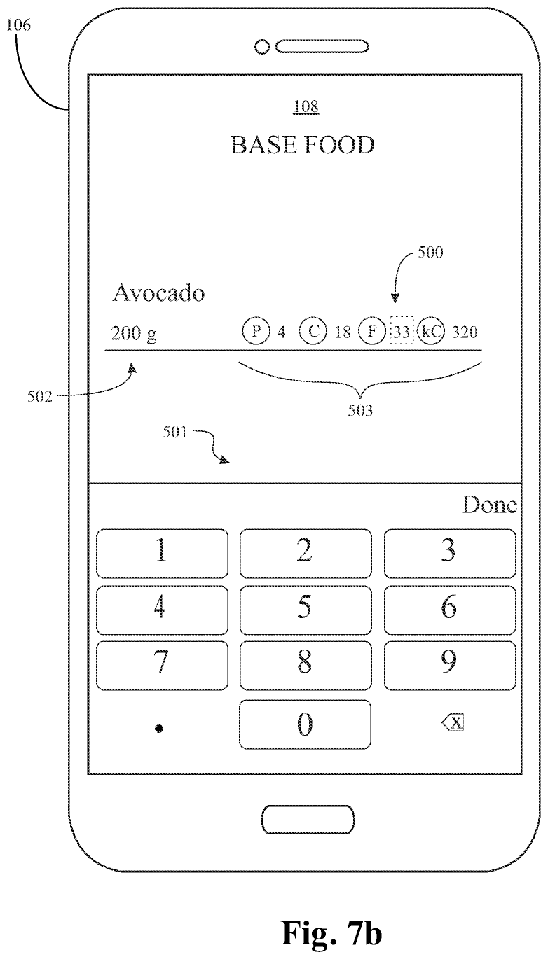

FIGS. 7a-b illustrate a graphical user interface 108 exemplary instance of a nutrient modification 500 for a base food element on a user device 106. Referring now to FIGS. 7a-b, in one embodiment, a user navigating the system's GUI showing a base food element, in this example, Avocado. Advantageously, the system enables the user to edit a base food element data such that any individual nutrient value may be adjusted without affecting any other nutrient values. For example, in the illustrated example, the user selects a nutrient modification 500 of a fat nutrient value and dynamically adjusts the value from “30” to “33” via the numerical keyboard 501. After this nutrient modification is confirmed, none of the plurality of nutrient values 503 are modified. Similarly, the serving size 502 is not modified. This provides several advantages, including but not limited to (a) enabling the user to correct nutrition values of base foods if they discover errors, (b) enables flexibility of nutrient value modifications without changing the serving size, and (c) enables the ability to update meal nutrient data elements when the base food element data is updated. Regarding advantage (a) presented above, the following are two examples of use cases. In one embodiment, the system addresses a scenario wherein a user queries a database for a specific food item from a retail establishment, such as Trader Joe's®. The system retrieves the item, but the displayed nutritional content, such as fat content, may be outdated due to recent updates by the retail establishment that have not yet been reflected in the database. In this instance, the user is provided with an option to edit the nutritional content to align it with the most current information. In another embodiment, the system accommodates unique dietary requirements by allowing the user to modify nutritional information for specific food items. For example, if a user adheres to a specialized diet that disregards the fat content in meats with a lean percentage greater than 90%, the system enables the user to locate an entry for a lean meat item and selectively remove the fat content from the displayed nutritional information, in accordance with the user's unique dietary rules.

Regarding these advantages, enabling the user to correct nutrition values of base foods if they discover errors, the base food data provides more accurate results, enables the users to adhere to their particular diet and eating habits, and goes beyond traditional nutrient value estimates enabling the user to dial in their nutritional consumption. Next, the flexibility of nutrient value modifications in base foods without changing the serving size of instances of the same food which have been previously added to meals or recipes is critical to accurately maintain a true volume of food consumed. It prevents unrealistic serving size consumption data, both in the planning phase and in the historical data. For example, referring to the previous example, if a user adjusted the fat nutrient value from “6.5” to “10”, and this adjusted the serving size, the serving size would adjust from “1 slice” to “1.54 slices.” For the planning phase and meal preparation, it is not helpful to have awkward serving sizes that are difficult to achieve, and from a historical data perspective, it would be inaccurate for changes to a base food's nutrients to modify the serving sizes of previously consumed foods. Finally, the system automatically updates meal nutrient data elements when the base food data is updated, which makes for more accurate meal planning and provides clear targets when combining multiple base food elements to create a meal. This also allows these base food data elements to be used in the future for additional meals, streamlining the process for nutrient tracking in the future, as the updated base food element data is saved in the data repository or local storage device of the computing device.

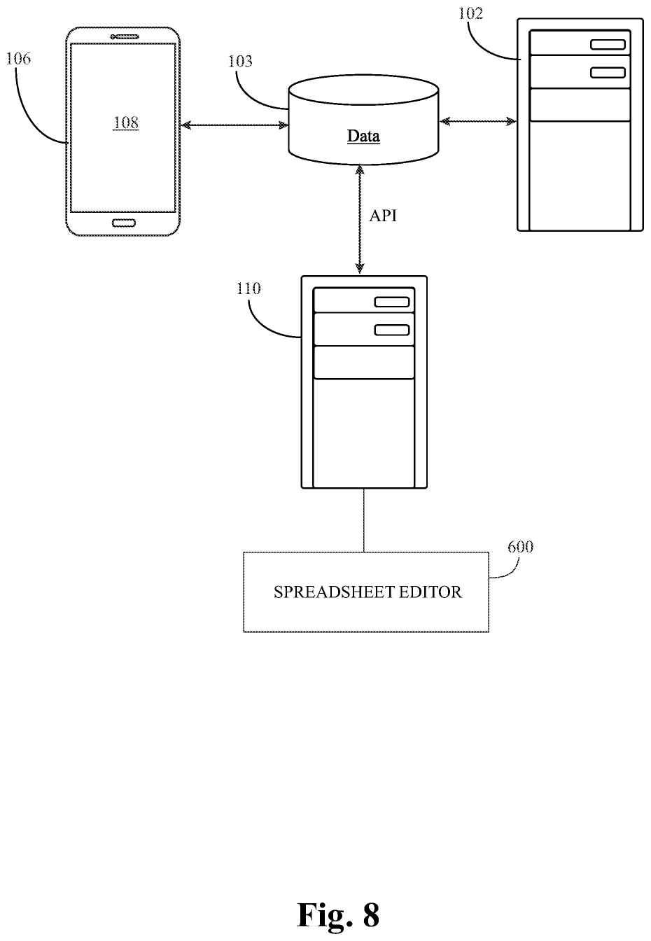

FIG. 8 is a system architectural diagram of the auto-calculating nutrient tracking system according to an embodiment of the present invention. Referring now to FIG. 8, a mobile device 106 having a graphical user interface (GUI) 108 is illustrated in communication with data repository 103, central server 102, and additional computing device 110, such as a desktop computer. The system allows integration with a spreadsheet editor 600, e.g. Google® sheets, such that using an application programming interface (API) can input data from the spreadsheet editor to the data repository 103 enabling the data to be utilized by the user, for instance base food element data. It should be noted that the integration may be performed by one computing device, such as mobile device 106, via a spreadsheet editor 600 provided on mobile device 106. This illustrates the integration of the system with other tools to provide ease of use and advantages to the users. This specific integration is also helpful in reverse, via exporting data from the system to the spreadsheet editor, enabling the user to provide further analysis of their historical data.

Although the invention has been described in considerable detail in language specific to structural features, it is to be understood that the invention defined in the appended claims is not necessarily limited to the specific features described. Rather, the specific features are disclosed as exemplary preferred forms of implementing the claimed invention. Stated otherwise, it is to be understood that the phraseology and terminology employed herein, as well as the abstract, are for the purpose of description and should not be regarded as limiting. Therefore, while exemplary illustrative embodiments of the invention have been described, numerous variations and alternative embodiments will occur to those skilled in the art. Such variations and alternative embodiments are contemplated, and can be made without departing from the spirit and scope of the invention.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

What is claimed is:

1. A nutrient tracking system for dynamically adjusting serving sizes and nutrient values, the system comprising:
an application software executable on one or more computing devices, wherein the application software executes a graphical user interface on the one or more computing devices;
a data repository for storing food data entries and user-defined nutrient targets, wherein the food data entries includes meals comprised of one or more food elements, each food element having a serving size and nutrient values for a plurality of nutrients;
a computational engine configured to:
receive user inputs for specific nutrient value targets within a food element of the one or more food elements;
calculate a scaling factor based on the user inputs;
adjust the serving size and nutrient values of the food element in real-time based on the calculated scaling factor;
recalculate the scaling factor when a user modifies a nutrient value within a food element; and,
update the graphical user interface with the updated nutrient values and serving size.

2. The nutrient tracking system of claim 1, wherein the plurality of nutrients include at least one of: protein, carbohydrates, fat, calories, cholesterol, fiber, water, sodium, saturated fat, sugar, unsaturated fat, vitamins, iron, potassium, calcium, and minerals.

3. The nutrient tracking system of claim 1, wherein the data repository is cloud-based, allowing for remote access and data synchronization across multiple devices.

4. The nutrient tracking system of claim 1, wherein a user, via the graphical user interface, may import food data entries from the cloud-based data repository.

5. The nutrient tracking system of claim 1, wherein the system provides a visual guide and numerical indication, via the graphical user interface, the progress of a user-defined target.

6. A nutrient tracking system for modifying base food element data, the system comprising:
an application software executable on one or more computing devices, wherein the application software executes a graphical user interface on the one or more computing devices;
a data repository for storing base food element data for a plurality of food elements, daily food data entries comprised of at least one food element of the plurality of food elements, and user-defined nutrient targets, wherein each base food element of the base food element data includes a serving size and nutrient values for a plurality of nutrients;
the graphical user interface configured to:
allow a user to select a base food element from the plurality of food elements;
enable the user to modify any individual nutrient value within the selected base food element;
wherein the modification is isolated to the individual nutrient value and does not affect other nutrient values within the selected base food element or its serving size; and,
a computational engine configured to:
receive user nutrient value inputs for specific nutrient targets within a food element of the plurality of food elements of a daily food data entry, wherein the food element has default nutrient values correlated to the base food element's modified nutrient values of the plurality of nutrients;
calculate a scaling factor based on the user inputs;
adjust the serving size and nutrient values of the food element in real-time based on the calculated scaling factor;
recalculate the scaling factor when a user modifies a nutrient value within a food element;
wherein the user interface is also configured to update the graphical user interface with the updated nutrient values and serving size;
and wherein said modification of any individual nutrient value of the daily food data entry does not affect other nutrient values within the selected base food element or its serving size.

7. The nutrient tracking system of claim 6, wherein the plurality of nutrients include at least one of: protein, carbohydrates, fat, calories, cholesterol, fiber, water, sodium, saturated fat, sugar, unsaturated fat, vitamins, iron, potassium, calcium, and minerals.

8. The nutrient tracking system of claim 6, wherein the data repository is cloud-based, allowing for remote access and data synchronization across multiple devices.

9. The nutrient tracking system of claim 6, wherein a user, via the graphical user interface, may import base food element data from the cloud-based data repository.

10. The nutrient tracking system of claim 6, wherein the system provides a visual guide and numerical indication, via the graphical user interface, the progress of a user-defined target.

11. A method for nutrient tracking and dynamic adjustment of serving sizes and nutrient values, the method comprising the steps of:
executing an application software on one or more computing devices, wherein the application software provides a graphical user interface;
storing food data entries and user-defined nutrient targets in a data repository, wherein the food data entries include meals comprised of one or more food elements, each food element having a serving size and nutrient values for a plurality of nutrients;
receiving, via the graphical user interface, user inputs for specific nutrient value targets within a food element of the one or more food elements;
calculating a scaling factor based on the user inputs;
adjusting the serving size and nutrient values of the food element in real-time based on the calculated scaling factor;
recalculating the scaling factor when a user modifies a nutrient value within a food element;
updating the graphical user interface with the updated nutrient values and serving size;
allowing a user to select a base food element from a plurality of food elements stored in the data repository;
enabling the user to modify any individual nutrient value within the selected base food element;
wherein the modification is isolated to the individual nutrient value and does not affect other nutrient values within the selected base food element or its serving size.
